(19) United States
(12) Patent Application Publication
(10) Pub. No.: US 2021/0139641 A1
(43) Pub. Date: May 13, 2021

(54) ACTIVE ESTER COMPOSITION AND SEMICONDUCTOR SEALING MATERIAL

(71) Applicant: DIC Corporation, Tokyo (JP)

(72) Inventors: Yutaka Satou, Ichihara-shi (JP); Akito Kawasaki, Ichihara-shi (JP)

(21) Appl. No.: 16/623,164

(22) PCT Filed: Jun. 5, 2018
(86) PCT No.: PCT/JP2018/021498

§ 371 Date: Dec. 16, 2019

(30) Foreign Application Priority Data
Jun. 28, 2017 (JP) 2017-126263

(51) Int. Cl. C08G 59/40; H01L 23/29; H05K 1/03

(52) U.S. Cl. CPC C08G 59/4071; H05K 2201/0145; H05K 1/0353; H01L 23/293

(57) ABSTRACT

There are provided an active ester composition that has high curability and that provides a cured product exhibiting excellent performance, such as in dielectric characteristics and heat resistance; a cured product of the composition; and a semiconductor sealing material and a printed wiring board that are formed from the composition. Specifically, an active ester composition containing an active ester compound and a benzoxazine compound as essential components is provided. Because the active ester composition containing the active ester compound and the benzoxazine compound as essential components is characterized by having high curability with a curing agent and providing a cured product exhibiting excellent performance, such as in dielectric characteristics and heat resistance, the active ester composition is suitable for use as a resin material for semiconductor sealing materials and printed wiring boards.

### TECHNICAL FIELD

The present invention relates to an active ester composition that has high curability and that provides a cured product exhibiting excellent performance, such as in dielectric characteristics and heat resistance, a cured product of the composition, and a semiconductor sealing material and a printed wiring board that are formed from the composition.

### BACKGROUND ART

In the technical field of insulating materials for use in, for example, semiconductors and multilayer printed wiring boards, as many types of electronic components become thinner and smaller, the development of new resin materials in line with such market trends has been required. For example, both a low dielectric constant and a low dielectric loss tangent are required for cured products to further reduce exothermic energy loss in response to the increased speed and frequency of signals. Furthermore, increasingly thinner semiconductors tend to contribute to decreased reliability due to warpage and distortion of the components. To suppress this, low curing shrinkage and a low linear expansion coefficient are also important.

A technology using, as an epoxy resin curing agent, di (α-naphthyl) isophthalate serving as a resin material that provides a cured product exhibiting a low dielectric constant and a low dielectric loss tangent is known (see PTL 1 below). Because di (α-naphthyl) isophthalate is in use as an epoxy resin curing agent, cured products of an epoxy resin composition described in PTL 1 certainly exhibit a low dielectric constant and a low dielectric loss tangent compared with the case where an existing epoxy resin curing agent, such as a novolac phenolic resin, is used. However, the epoxy resin composition described in PTL 1 has low curability and requires curing at a high temperature and for a long time, resulting in a problem of decreased productivity and energy costs for industrial use.

### CITATION LIST

### Patent Literature

PTL 1: Japanese Unexamined Patent Application Publication No. 2003-82063

### SUMMARY OF INVENTION

### Technical Problem

Thus, an object of the present invention is to provide an active ester composition that has high curability and that provides a cured product exhibiting excellent performance, such as in dielectric characteristics and heat resistance, a cured product of the composition, and a semiconductor sealing material and a printed wiring board that are formed from the composition.

### Solution to Problem

As a result of intensive research in an effort to achieve the object described above, the present inventors have found that a composition containing an active esterified product and a benzoxazine compound has high curability and that a cured product of the composition exhibits excellent performance, such as in dielectric characteristics and heat resistance, and have completed the invention.

Thus, the present invention relates to an active ester composition containing an active ester compound (A) and a benzoxazine compound (B) as essential components.

The present invention further relates to a curable composition containing the active ester composition and a curing agent.

The present invention further relates to a cured product of the curable composition.

The present invention further relates to a semiconductor sealing material formed from the curable composition.

The present invention further relates to a printed wiring board formed from the curable composition.

### Advantageous Effects of Invention

According to the present invention, an active ester composition that has high curability and that provides a cured product exhibiting excellent performance, such as in dielectric characteristics and heat resistance, a cured product of the composition, and a semiconductor sealing material and a printed wiring board that are formed from the composition can be provided.

### DESCRIPTION OF EMBODIMENTS

Hereafter, the present invention will be described in detail.

The active ester composition according to the present invention is characterized by containing an active ester compound (A) and a benzoxazine compound (B) as essential components.

The active ester compound (A) may have any specific structure as long as the molecular structure of the compound has an aromatic polyester structure. Furthermore, the active ester compound (A) may have any molecular weight and may be a single molecular weight compound or an oligomer or polymer having a molecular weight distribution. Specific examples of the active ester compound (A) include (A1) to (A4) described below. These are merely examples of the active ester compound (A), and the active ester compound (A) according to the present invention is not limited thereto. Furthermore, the active ester compound (A) may be used alone or in combination of two or more thereof.
An active ester compound (A1): an esterified product of a compound having one phenolic hydroxyl group in a molecular structure (a1) and an aromatic polycarboxylic acid or an acid halide thereof (a2)
An active ester compound (A2): an esterified product of a compound having two or more phenolic hydroxyl groups in a molecular structure (a3) and an aromatic monocarboxylic acid or an acid halide thereof (a4)
An active ester compound (A3): an esterified product of a compound having one phenolic hydroxyl group in a molecular structure (a1), an aromatic polycarboxylic acid or an acid halide thereof (a2), and a compound having two or more phenolic hydroxyl groups in a molecular structure (a3)
An active ester compound (A4): an esterified product of an aromatic polycarboxylic acid or an acid halide thereof (a2), a compound having two or more phenolic hydroxyl groups in a molecular structure (a3), and an aromatic monocarboxylic acid or an acid halide thereof (a4)

Specific examples of the compound having one phenolic hydroxyl group in a molecular structure (a1) include phenol or a phenolic compound having one or more substituents on the aromatic nucleus of phenol, naphthol or a naphthol compound having one or more substituents on an aromatic nucleus of naphthol, and anthracenol or an anthracenol compound having one or more substituents on an aromatic nucleus of anthracenol. Examples of the substituent on an aromatic nucleus include alkyl groups such as a methyl group, an ethyl group, a propyl group, a butyl group, a pentyl group, a hexyl group, a cyclohexyl group, a heptyl group, an octyl group, and a nonyl group; alkoxy groups such as a methoxy group, an ethoxy group, a propyloxy group, and a butoxy group; a moiety containing an unsaturated group such as a vinyl group, a vinyloxy group, an allyl group, an allyloxy group, a propargyl group, or a propargyloxy group; halogen atoms such as a fluorine atom, a chlorine atom, and a bromine atom; a phenyl group, a naphthyl group, an anthryl group, and an aryl group in which an aromatic nucleus of the foregoing is substituted with the alkyl group, the alkoxy group, the moiety containing an unsaturated group, the halogen atom, or the like; and a phenylmethyl group, a phenylethyl group, a naphthylmethyl group, a naphthylethyl group, and an aralkyl group in which an aromatic nucleus of the foregoing is substituted with the alkyl group, the alkoxy group, the moiety containing an unsaturated group, the halogen atom, or the like. The above-described compounds may be used alone or in combination of two or more thereof.

Among the compounds, in view of obtaining a cured product exhibiting excellent performance, such as in dielectric characteristics and heat resistance, a phenolic compound or a naphthol compound is preferable, and phenol, naphthol, or either of these having one or two aliphatic hydrocarbon groups or aryl groups on an aromatic nucleus thereof is more preferable.

Examples of the aromatic polycarboxylic acid or an acid halide thereof (a2) include benzenedicarboxylic acids such as isophthalic acid and terephthalic acid, benzenetricarboxylic acids such as trimellitic acid, naphthalene dicarboxylic acids such as naphthalene-1,4-dicarboxylic acid, naphthalene-2,3-dicarboxylic acid, naphthalene-2,6-dicarboxylic acid, and naphthalene-2,7-dicarboxylic acid; acid halides thereof; and compounds in which an aromatic nucleus of the foregoing is substituted with the alkyl group, the alkoxy group, the moiety containing an unsaturated group, the halogen atom, or the like. Examples of the acid halides include acid chloride, acid bromide, acid fluoride, and acid iodide. These may be used individually or in combination of two or more thereof. Among these, in view of obtaining a cured product exhibiting excellent performance, such as in dielectric characteristics and heat resistance, benzenedicarboxylic acids such as isophthalic acid and terephthalic acid and acid halides thereof are preferable.

Examples of the compound having two or more phenolic hydroxyl groups in a molecular structure (a3) include dihydroxybenzene, dihydroxynaphthalene, dihydroxyanthracene, biphenol, and bisphenol; and compounds having one or more substituents on aromatic nuclei of these compounds. In addition, the examples also include a novolac resin formed from one or more of the compounds having one phenolic hydroxyl group in a molecular structure (a1) which serve as reaction raw materials therefor, and a reaction product of one or more of the compounds having one phenolic hydroxyl group in a molecular structure (a1) and a compound (x) represented by any of Structural Formulas (x-1) to (x-5) below which serve as essential reaction raw materials therefor.

[chemical structure]

[wherein h represents 0 or 1. R²'s each independently represent an alkyl group, an alkoxy group, a moiety containing an unsaturated group, a halogen atom, an aryl group, or an aralkyl group, and i represents 0 or an integer of 1 to 4. Z represents a vinyl group, a halomethyl group, a hydroxymethyl group, or an alkyloxy methyl group. Y represents an alkylene group of 1 to 4 carbon atoms, an oxygen atom, a sulfur atom, or a carbonyl group. j represents an integer of 1 to 4.]

The compounds having two or more phenolic hydroxyl groups in a molecular structure (a3) may be used individually or in combination of two or more thereof. Among them, in view of obtaining a cured product exhibiting excellent performance, such as in dielectric characteristics and heat resistance, a reaction product of one or more of the compounds having one phenolic hydroxyl group in a molecular structure (a1) and the compound (x) represented by any of Structural Formulas (x-1) to (x-4) above which serve as essential reaction raw materials therefor is preferable. A reaction of the compound having one phenolic hydroxyl group in a molecular structure (a1) and the compound (x) can be performed using a method of heating and agitating these under an acid catalyst condition at a temperature of about 80° C. to 180° C.

Examples of the aromatic monocarboxylic acid or an acid halide thereof (a4) include benzoic acid, benzoyl halide, and compounds in which an aromatic nucleus of the foregoing is substituted with the alkyl group, the alkoxy group, the moiety containing an unsaturated group, the halogen atom, or the like. These may be used individually or in combination of two or more thereof.

The active ester compound (A) may be produced, for example, using a method in which individual reaction raw materials are mixed and agitated in the presence of an alkaline catalyst and at a temperature of about 40° C. to 65° C. As needed, the reaction may be performed in an organic solvent. After the reaction, the reaction product may be purified by, for example, rinsing or reprecipitation.

Examples of the alkaline catalyst include sodium hydroxide, potassium hydroxide, triethylamine, and pyridine. These may be used individually or in combination of two or more thereof. These may also be used as an about 3.0 to 30% aqueous solution. Among them, sodium hydroxide or potassium hydroxide having high catalytic ability is preferable.

Examples of the organic solvent include ketone solvents such as acetone, methyl ethyl ketone, and cyclohexanone; acetate solvents such as ethyl acetate, butyl acetate, cellosolve acetate, propylene glycol monomethyl ether acetate, and carbitol acetate; carbitol solvents such as cellosolve and butyl carbitol; aromatic hydrocarbon solvents such as toluene and xylene; and dimethylformamide, dimethylacetamide, N-methyl pyrrolidone, and the like. These may be used individually or as a mixed solvent of two or more thereof.

The reaction ratio of the individual reaction raw materials is appropriately adjusted depending on, for example, the desired physical properties of the active ester compound (A), but is particularly preferably as follows.

In the production of the active ester compound (A1), in view of obtaining the target active ester compound (A1) at a high yield, the reaction ratio of the compound having one phenolic hydroxyl group in a molecular structure (a1) and the aromatic polycarboxylic acid or an acid halide thereof (a2) is preferably a ratio of 0.95 to 1.05 moles of the compound having one phenolic hydroxyl group in a molecular structure (a1) to 1 mole of the total amount of carboxyl groups or acid halide groups contained in the aromatic polycarboxylic acid or an acid halide thereof (a2).

In the production of the active ester compound (A2), in view of obtaining the target active ester compound (A2) at a high yield, the reaction ratio of the esterified product of the compound having two or more phenolic hydroxyl groups in a molecular structure (a3) and the aromatic monocarboxylic acid or an acid halide thereof (a4) is preferably a ratio of 0.95 to 1.05 moles of the aromatic monocarboxylic acid or an acid halide thereof (a4) to 1 mole of the total amount of phenolic hydroxyl groups contained in the compound having two or more phenolic hydroxyl groups in a molecular structure (a3).

In the production of the active ester compound (A3), the reaction ratio of the compound having one phenolic hydroxyl group in a molecular structure (a1), the aromatic polycarboxylic acid or an acid halide thereof (a2), and the compound having two or more phenolic hydroxyl groups in a molecular structure (a3) is such that the ratio of the number of moles of hydroxyl groups in the compound having one phenolic hydroxyl group in a molecular structure (a1) to the number of moles of hydroxyl groups in the compound having two or more phenolic hydroxyl groups in a molecular structure (a3) is preferably 95/5 to 25/75, and more preferably 90/10 to 70/30. Furthermore, the total amount of hydroxyl groups contained in the compound having one phenolic hydroxyl group in a molecular structure (a1) and hydroxyl groups contained in the compound having two or more phenolic hydroxyl groups in a molecular structure (a3) is preferably in the range of 0.95 to 1.05 moles with respect to 1 mole of the total amount of carboxyl groups or acid halide groups contained in the aromatic polycarboxylic acid or an acid halide thereof (a2). Furthermore, in the production of the active ester compound (A3), the active ester compound (A3) may be produced as a mixture of the active ester compounds (A1) and (A3), by appropriately adjusting the reaction ratio of the individual components.

In the production of the active ester compound (A4), the reaction ratio of the aromatic polycarboxylic acid or an acid halide thereof (a2), the compound having two or more phenolic hydroxyl groups in a molecular structure (a3), and the aromatic monocarboxylic acid or an acid halide thereof (a4) is such that the ratio of the total amount of carboxyl groups or acid halide groups contained in the aromatic monocarboxylic acid or an acid halide thereof (a4) to the total amount of carboxyl groups or acid halide groups contained in the aromatic polycarboxylic acid or an acid halide thereof (a2) is preferably 95/5 to 25/75, and more preferably 90/10 to 70/30. Furthermore, the total amount of carboxyl groups or acid halide groups contained in the aromatic polycarboxylic acid or an acid halide thereof (a2) and in the aromatic monocarboxylic acid or an acid halide thereof (a4) is preferably in the range of 0.95 to 1.05 with respect to 1 mole of hydroxyl groups contained in the compound having two or more phenolic hydroxyl groups in a molecular structure (a3). Furthermore, in the production of the active ester compound (A4), the active ester compound (A4) may be produced as a mixture of the active ester compounds (A2) and (A4), by appropriately adjusting the reaction ratio of the individual components.

The melt viscosity of the active ester compounds (A1) and (A2) at 150° C. is preferably in the range of 0.01 to 5 dPa·s. In the present invention, the melt viscosity at 150° C. is measured with an ICI viscometer in accordance with ASTM D4287.

The softening point of the active ester compounds (A3) and (A4) measured in accordance with JIS K7234 is preferably in the range of 80° C. to 180° C., and more preferably in the range of 85° C. to 160° C.

Furthermore, when a mixture of a plurality of the active ester compounds (A) is in use, the melt viscosity of the active ester compounds (A) as a whole at 150° C. is preferably in the range of 0.01 to 50 dPa·s., and more preferably in the range of 0.01 to 10 dPa·s. The melt viscosity at 150° C. is measured with an ICI viscometer in accordance of ASTM D4287.

Among the active ester compounds (A1) to (A4), the active ester compound (A1) or (A2) is preferable, and the active ester compound (A1) is particularly preferable, because a cured product thereof exhibits particularly excellent dielectric characteristics and particularly excellent heat resistance and the active ester compound has low melt viscosity. The proportion of the active ester compound (A1) or (A2) contained in the active ester compound (A) is preferably 30% or more, more preferably 50% or more, and particularly preferably 60% or more. In the present invention, the proportion of the active ester compounds (A1) and (A2) contained in the active ester compound (A) is calculated in accordance with an area ratio in a GPC chart measured under the following conditions.

Measurement Device: “HLC-8320 GPC” manufactured by Tosoh Corporation

Columns: guard column “HXL-L” manufactured by Tosoh Corporation

+“TSK-GEL G4000HXL” manufactured by Tosoh Corporation

+“TSK-GEL G3000HXL” manufactured by Tosoh Corporation

+“TSK-GEL G2000HXL” manufactured by Tosoh Corporation

+“TSK-GEL G2000HXL” manufactured by Tosoh Corporation

Detector: RI (differential refractometer)

Data Processing: “GPC WorkStation EcoSEC-WorkStation” manufactured by Tosoh Corporation

Measurement Conditions:
Column Temperature 40° C.
Developing Solvent Tetrahydrofuran
Flow Rate 1.0 ml/min

Standard: The following monodisperse polystyrenes having known molecular weights were used in accordance with the “GPC-8320” measurement manual.

(Polystyrenes Used)
“A-500” manufactured by Tosoh Corporation
“A-1000” manufactured by Tosoh Corporation
“A-2500” manufactured by Tosoh Corporation
“A-5000” manufactured by Tosoh Corporation
“F-1” manufactured by Tosoh Corporation
“F-2” manufactured by Tosoh Corporation
“F-4” manufactured by Tosoh Corporation
“F-10” manufactured by Tosoh Corporation
“F-20” manufactured by Tosoh Corporation
“F-40” manufactured by Tosoh Corporation
“F-80” manufactured by Tosoh Corporation
“F-128” manufactured by Tosoh Corporation

Sample: A 1.0% by mass (in terms of resin solid content) aliquot of tetrahydrofuran solution filtered with a micro filter (50 μl)

The specific structure, the molecular weight, and the like of the benzoxazine compound (B) are not particularly limited as long as the compound has one or more benzoxazine ring structures in a molecular structure, and various compounds may be used. Examples of the benzoxazine compound (B) include a reaction product of an aromatic amine compound (b1), a phenolic hydroxyl group-containing compound (b2), and formaldehyde which serve as essential reaction raw materials therefor.

The aromatic amine compound (b1) may be phenylamine, phenylenediamine, diaminodiphenyl alkane, and diaminodiphenyl sulfone, compounds having one or more substituents on aromatic nuclei of these, or the like. Examples of substituents on the aromatic nuclei include alkyl groups such as a methyl group, an ethyl group, a propyl group, a butyl group, a pentyl group, a hexyl group, a cyclohexyl group, a heptyl group, an octyl group, and a nonyl group; alkoxy groups such as a methoxy group, an ethoxy group, a propyloxy group, and a butoxy group; a moiety containing an unsaturated group such as a vinyl group, a vinyloxy group, an allyl group, an allyloxy group, a propargyl group, or a propargyloxy group; halogen atoms such as a fluorine atom, a chlorine atom, and a bromine atom; a phenyl group, a naphthyl group, an anthryl group, and an aryl group in which an aromatic nucleus of the foregoing is substituted with the alkyl group, the alkoxy group, the moiety containing an unsaturated group, the halogen atom, or the like; and a phenylmethyl group, a phenylethyl group, a naphthylmethyl group, a naphthylethyl group, and an aralkyl group in which an aromatic nucleus of the foregoing is substituted with the alkyl group, the alkoxy group, the moiety containing an unsaturated group, the halogen atom, or the like. The above-described compounds may be used individually or in combination of two or more thereof. Among these, phenylamine, phenylenediamine, diaminodiphenylmethane, or a compound having one or more moieties containing an unsaturated group on an aromatic nucleus of any of these is preferable, because of the capability to turn into an active ester composition having high curability and exhibiting an excellent balance between viscosity and heat resistance.

Examples of the phenolic hydroxyl group-containing compound (b2) include the compound having one phenolic hydroxyl group in a molecular structure (a1) or the compound having two or more phenolic hydroxyl groups in a molecular structure (a3) exemplified above. These may be used individually or in combination of two or more thereof. Among these, phenol, naphthol, or a compound having one or more moieties containing an unsaturated group on an aromatic nucleus of any of these is preferable because of the capability to turn into an active ester composition having high curability and exhibiting an excellent balance between viscosity and heat resistance.

The benzoxazine compound (B) may be produced, for example, using a method in which reaction raw materials are mixed and agitated at a temperature of about 50° C. to 100° C. As needed, the reaction may be performed in an organic solvent. After the reaction, the reaction product may be purified by, for example, rinsing or reprecipitation.

Examples of the organic solvent include ketone solvents such as acetone, methyl ethyl ketone, and cyclohexanone; acetate solvents such as ethyl acetate, butyl acetate, cellosolve acetate, propylene glycol monomethyl ether acetate, and carbitol acetate; carbitol solvents such as cellosolve and butyl carbitol, aromatic hydrocarbon solvents such as toluene and xylene; and dimethylformamide, dimethylacetamide, N-methyl pyrrolidone, and the like. These may be used individually or as a mixed solvent of two or more thereof.

The reaction ratio of the aromatic amine compound (b1), the phenolic hydroxyl group-containing compound (b2), and formaldehyde is appropriately adjusted depending on, for example, the desired physical properties of the obtained benzoxazine compound (B), but is particularly preferably as follows. The number of moles of hydroxyl groups contained in the phenolic hydroxyl group-containing compound (b2) is preferably in the range of 0.95 to 1.05 with respect to 1 mole of amino groups contained in the aromatic amine compound (b1). Furthermore, the amount of formaldehyde for use is preferably in the range of 1.95 to 2.05 moles with respect to the total amount of amino groups contained in the aromatic amine compound (b2) and hydroxyl groups contained in the phenolic hydroxyl group-containing compound (b2).

In the active ester composition according to the present invention, the mixing ratio of the active ester compound (A) and the benzoxazine compound (B) is appropriately adjusted depending on, for example, the desired curability and physical properties of the cured product, but the content of the benzoxazine compound (B) is preferably in the range of 0.1 to 500 parts by mass, more preferably in the range of 10 to 400 parts by mass, and particularly preferably in the range of 10 to 90 parts by mass with respect to the 100 parts by mass of the active ester compound (A) particularly in view of an excellent balance between curability and physical properties of the cured product.

The curable composition according to the present invention contains the active ester composition and a curing agent. The curing agent may be any compound that is capable of reacting with the active ester composition according to the present invention, and the compound for use may vary without any particular limitation. One example of the curing agents is an epoxy resin. Examples of the epoxy resin include polyglycidyl ether, which is one of the compounds having two or more phenolic hydroxyl groups in a molecular structure (a3).

In the curable composition according to the present invention, the mixing ratio of the active ester composition and the curing agent is not particularly limited, and may be appropriately adjusted depending on, for example, the desired performance of the cured product. One example of the mixing ratio when an epoxy resin as a curing agent is in use is preferably a ratio of 0.7 to 1.5 moles of the total amount of functional groups contained in the active ester composition to 1 mole of the total amount of epoxy groups contained in the epoxy resin. In the present invention, the “functional groups contained in the active ester composition” refers to ester bond moieties and benzoxazine ring structures contained in the active ester composition. Furthermore, the equivalent of the functional groups contained in the active ester composition is calculated in accordance with the amount of the reaction raw materials charged.

The curable composition according to the present invention may further contain a curing accelerator. Examples of the curing accelerator include phosphorus compounds, tertiary amines, imidazole compounds, pyridine compounds, organic acid metal salts, Lewis acids, and amine complex salts. Among them, because of exhibiting excellent curability, excellent heat resistance, excellent dielectric characteristics, excellent moisture absorption resistance, and the like, triphenylphosphine is preferable among phosphorus compounds, 1,8-diazabicyclo[5.4.0]undecene (DBU) is preferable among tertiary amines, 2-ethyl-4-methylimidazole is preferable among imidazole compounds, and 4-dimethylaminopyridine and 2-phenylimidazole are preferable among pyridine compounds. The amount of such a curing accelerator added is preferably in the range of 0.01 to 15% by mass with respect to the 100 parts by mass of the curable composition.

The curable composition according to the present invention may further contain other resin components. Examples of such other resin components include phenolic hydroxyl group-containing compounds such as the compound having two or more phenolic hydroxyl groups in a molecular structure (a3); amine compounds such as diaminodiphenylmethane, diethylenetriamine, triethylenetetramine, diaminodiphenyl sulfone, isophoronediamine, imidazole, a BF₃-amine complex, and a guanidine derivative; amide compounds such as dicyandiamide and a polyamide resin synthesized from a dimer of linolenic acid and ethylenediamine; acid anhydrides such as phthalic anhydride, trimellitic anhydride, pyromellitic anhydride, maleic anhydride, tetrahydrophthalic anhydride, methyltetrahydrophthalic anhydride, methyl nadic anhydride, hexahydrophthalic anhydride, and methylhexahydrophthalic anhydride; a cyanic acid ester resin; a bismaleimide resin; a styrene-maleic anhydride resin; allyl group-containing resins typified by diallylbisphenol and triallyl isocyanurate; and a polyphosphoric acid ester and a phosphoric acid ester-carbonate copolymer. These may be used individually or in combination of two or more thereof.

The mixing ratio of these other resin components is not particularly limited and may be appropriately adjusted depending on, for example, the desired performance of the cured product. One example of the mixing ratio is preferably the use in the range of 1 to 50% by mass in the curable composition according to the present invention.

The curable composition according to the present invention may contain various additives such as a flame retardant, an inorganic filler, a silane coupling agent, a release agent, a pigment, an emulsifier, and the like.

Examples of the flame retardant include inorganic phosphorus compounds such as red phosphorus, ammonium phosphates such as monoammonium phosphate, diammonium phosphate, triammonium phosphate, and ammonium polyphosphate, and amide phosphate; organic phosphorous compounds such as a phosphoric acid ester compound, a phosphonic acid compound, a phosphinic acid compound, a phosphine oxide compound, a phosphorane compound, an organic nitrogen-containing phosphorus compound, cyclic organic phosphorus compounds such as 9,10-dihydro-9-oxa-10-phosphaphenanthrene-10-oxide, 10-(2,5-dihydroxyphenyl)-10H-9-oxa-10-phosphaphenanthrene-10-oxide, 10-(2,7-dihydroxynaphthyl)-10H-9-oxa-10-phosphaphenanthrene-10-oxide, and derivatives obtained by reacting the cyclic organic phosphorous compounds with a compound such as an epoxy resin or a phenolic resin; nitrogen-based flame retardants such as a triazine compound, a cyanuric acid compound, an isocyanuric acid compound, and phenothiazine; silicone-based flame retardants such as silicone oil, silicone rubber, and a silicone resin; and inorganic flame retardants such as metal hydroxide, metal oxide, a metal carbonate compound, metal powder, a boron compound, and low melting point glass. When any of these flame retardants is in use, the flame retardant is preferably used in the range of 0.1 to 20% by mass in the curable composition.

The inorganic filler is mixed when, for example, the curable composition according to the present invention is used for a semiconductor sealing material. Examples of the inorganic filler include molten silica, crystalline silica, alumina, silicon nitride, and aluminum hydroxide. Among them, molten silica is preferable because molten silica enables a greater amount of the inorganic filler to be mixed. The molten silica for use may be crushed or spherical, but the main use of spherical silica is preferable to increase the mixing amount of the molten silica and to suppress the increase of melt viscosity of the curable composition. Furthermore, an appropriate adjustment of the particle size distribution of the spherical silica is preferable to increase the mixing amount of the spherical silica. The filling rate thereof is preferably in the range of 0.5 to 95 parts by mass being mixed with respect to 100 parts by mass of the curable composition.

In addition, when the curable composition according to the present invention is in use for a conductive paste and the like, a conductive filler such as silver powder or copper powder may be used.

The active ester composition and the curable composition using the active ester composition according to the present invention are characterized by having high curability and providing a cured product exhibiting excellent physical properties, such as dielectric characteristics and heat resistance. In addition, the active ester composition and the curable composition using the active ester composition according to the present invention exhibit sufficiently high general performance required for resin materials, such as solubility in a general organic solvent and storage stability. Accordingly, the active ester composition and the curable composition using the active ester composition according to the present invention are widely applicable not only to electronic materials such as semiconductor sealing materials, printed wiring boards and resist materials, but also to, for example, paints, adhesives, and molded products.

When the curable composition according to the present invention is in use for a semiconductor sealing material, the mixing of an inorganic filler is generally preferable. A semiconductor sealing material may be prepared, by mixing the mixture using, for example, an extruder, a kneader, or a roller. Examples of methods for molding a semiconductor package using the obtained semiconductor sealing material include a method in which the semiconductor sealing material is molded with a cast, a transfer molding machine, or an injection molding machine and is heated at a temperature of 50° C. to 200° C. for 2 to 10 hours. A semiconductor device which is a molded article can be obtained using such a method.

When the curable composition according to the present invention is in use for printed wiring boards or build-up adhesive films, the composition is generally preferably mixed and diluted with an organic solvent. The organic solvent may be methyl ethyl ketone, acetone, dimethylformamide, methyl isobutyl ketone, methoxypropanol, cyclohexanone, methyl cellosolve, ethyl diglycol acetate, or propylene glycol monomethyl ether acetate. The type and the mixing amount of the organic solvent may be appropriately adjusted depending on the usage environment of the curable composition. However, for example, when used for a printed wiring board, a polar solvent having a boiling point of 160° C. or less, such as methyl ethyl ketone, acetone, or dimethylformamide, is preferable and the polar solvent is preferably used with the ratio of a non-volatile content being 40 to 80% by mass. When used for a build-up adhesive film, it is preferable to use ketone solvents such as acetone, methyl ethyl ketone, and cyclohexanone; acetate solvents such as ethyl acetate, butyl acetate, cellosolve acetate, propylene glycol monomethyl ether acetate, and carbitol acetate; carbitol solvents such as cellosolve and butyl carbitol, aromatic hydrocarbon solvents such as toluene and xylene; and dimethylformamide, dimethylacetamide, N-methylpyrrolidone, and the like. Any of these is preferably prepared with the ratio of a non-volatile content being 30 to 60% by mass.

Furthermore, examples of the method for manufacturing printed wiring boards using the curable composition according to the present invention include a method in which a reinforcement was impregnated with the curable composition and the curable composition was cured to obtain a prepreg, and the prepreg is placed on and thermally press-bonded with copper foil. The reinforcement may be paper, glass cloth, glass nonwoven cloth, aramid paper, aramid cloth, glass matting, or glass roving cloth. The impregnation amount of the curable composition is not particularly limited, but the prepreg is typically preferably prepared so as to have a resin content of 20 to 60% by mass.

### EXAMPLES

Hereafter, the present invention will be described in further detail with reference to Examples and Comparative Examples. The description of “parts” and “%” in the Examples are on a mass basis unless otherwise indicated.

In the present Examples, the melt viscosity of an active ester compound (A) is measured at 150° C. in accordance with ASTM D4287 using an ICI viscometer.

In the Examples, the number average molecular weight (Mn) of the raw material phenolic compound forming an active ester composition (A-2) was measured by GPC under the following conditions. Furthermore, the proportion of individual components contained in the active ester composition (A-2) was calculated in accordance with an area ratio in a GPC chart measured under the following conditions.

Measurement Device: “HLC-8320 GPC” manufactured by Tosoh Corporation

Columns: guard column “HXL-L” manufactured by Tosoh Corporation

+“TSK-GEL G4000HXL” manufactured by Tosoh Corporation

+“TSK-GEL G3000HXL” manufactured by Tosoh Corporation

+“TSK-GEL G2000HXL” manufactured by Tosoh Corporation

+“TSK-GEL G2000HXL” manufactured by Tosoh Corporation

Detector: RI (differential refractometer)

Data Processing: “GPC WorkStation EcoSEC-WorkStation” manufactured by Tosoh Corporation

Measurement Conditions:
Column Temperature 40° C.
Developing Solvent Tetrahydrofuran
Flow Rate 1.0 ml/min

Standard: The following monodisperse polystyrenes having known molecular weights were used in accordance with the “GPC-8320” measurement manual.

(Polystyrenes Used)
“A-500” manufactured by Tosoh Corporation
“A-1000” manufactured by Tosoh Corporation
“A-2500” manufactured by Tosoh Corporation
“A-5000” manufactured by Tosoh Corporation
“F-1” manufactured by Tosoh Corporation
“F-2” manufactured by Tosoh Corporation
“F-4” manufactured by Tosoh Corporation
“F-10” manufactured by Tosoh Corporation
“F-20” manufactured by Tosoh Corporation
“F-40” manufactured by Tosoh Corporation
“F-80” manufactured by Tosoh Corporation
“F-128” manufactured by Tosoh Corporation

Sample: A 1.0% by mass (in terms of resin solid content) aliquot of tetrahydrofuran solution filtered with a micro filter (50 μl)

### Production Example 1

### Production of Active Ester Compound (A-1)

Into a flask equipped with a thermometer, a dropping funnel, a condenser, a distillation column, and an agitator, 202 g of isophthalic acid chloride and 1250 g of toluene were charged, and the content was dissolved under reduced pressure in the system purged with nitrogen. Next, 288 g of 1-naphthol was charged and dissolved under reduced pressure in the system purged with nitrogen. Furthermore, 0.6 g of tetrabutylammonium bromide was added thereto under purging with nitrogen gas while the temperature inside the system was controlled to 60° C. or less, and 400 g of a 20% aqueous solution of sodium hydroxide was added dropwise to the mixture over 3 hours. After the dropwise addition was complete, agitation was continued for 1 hour for a reaction. After the reaction was complete, the reaction mixture was left to stand to be separated into phases, and the water layer was removed. Water was added to the remaining organic layer and was agitated and mixed for about 15 minutes. The mixture was then left to stand to be separated into phases, and the water layer was removed. This process was repeated until the pH of the water layer reached 7, and the moisture and toluene were removed by dehydration using a decanter to obtain an active ester compound (A-1). The melt viscosity of the active ester compound (A-1) was 0.6 dPa·s.

### Production Example 2

### Production of Active Ester (A-2)

Into a flask equipped with a thermometer, a dropping funnel, a cooling column, a fractionating column, and an agitator, 202 g of isophthalic acid chloride and 1250 g of toluene were charged, and the content was dissolved under reduced pressure in the system purged with nitrogen. Next, 247 g of 1-naphthol and 47 g of a dicyclopentadiene adduct phenolic compound (which is represented by Structural Formula below and of which the average t value calculated in accordance with the number average molecular weight (Mn) was 0.2, a hydroxyl group equivalent of 166.6 g/eq) were dissolved under reduced pressure in the system purged with nitrogen. Furthermore, 0.6 g of tetrabutylammonium bromide was added thereto under purging with nitrogen gas while the temperature inside the system was controlled to 60° C. or less, and 400 g of a 20% aqueous solution of sodium hydroxide was added dropwise to the mixture over 3 hours. After the dropwise addition was complete, agitation was continued for 1 hour for a reaction. After the reaction was complete, the reaction mixture was left to stand to be separated into phases, and the water layer was removed. Water was added to the remaining organic layer and was agitated and mixed for about 15 minutes. The mixture was then left to stand to be separated into phases, and the water layer was removed. This process was repeated until the pH of the water layer reached 7, and the moisture and toluene were removed by dehydration using a decanter to obtain an active ester (A-2). The melt viscosity of the active ester (A-2) was 2.5 dPa·s. Furthermore, the content of the component corresponding to the active ester compound (A1) in the active ester (A-2) was 73% in the calculation in accordance with an area ratio in a GPC chart.

[chemical structure]

(wherein n is 0 or 1, and t is 0 or an integer of 1 or more.)

### Production Example 3

### Production of Benzoxazine Compound (B-1)

Into a 4-neck flask equipped with a dropping funnel, a thermometer, an agitating device, a heating device, and a cooling reflux column, 147.2 g of 4-propargyloxyaniline and 148.2 g of 4-propargyloxyphenol were charged under purging with nitrogen gas and dissolved in 750 g of toluene. Next, 63.9 g of 94% paraformaldehyde was added thereto and was heated to 80° C. while agitating. The agitation was performed at 80° C. for 7 hours. The reaction mixture was then moved into a separatory funnel and a water layer was removed. The solvent was removed from the organic layer under reduced pressure while heating to obtain 239 g of a benzoxazine compound (B-1). The melt viscosity of the benzoxazine compound (B-1) was 0.1 dPa·s.

In addition, each of the compounds used in the Examples and Comparative Examples in the present application are as follows.
Benzoxazine compound (B-2): “Benzoxazine P-d type” manufactured by Shikoku Chemicals Corporation, represented by Structural Formula (B-2) below.
Epoxy Resin: “N-665-EXP-S” manufactured by DIC Corporation, cresol novolac epoxy resin, an epoxy group equivalent of 202 g/eq.

[chemical structure]

### Examples 1-5 and Comparative Examples 1,2

Individual components were mixed at the ratios given in Table 1 below and a curable composition was obtained. Each evaluation test was conducted on the obtained curable composition in the following manner. The results are given in Table 1.

### Measurement of ICI Viscosity

The melt viscosity of the curable composition where components except for triphenylphosphine were mixed was measured at 150° C. in accordance of ASTM D4287 using an ICI viscometer.

### Measurement of Gelling Time

After mixing the individual components at the ratios given in Table 1, 0.15 g of the curable composition was placed on a hotplate heated to 185° C., and the time required for the curable composition to gel was measured while agitating using a spatula. The same operation was repeated three times and an averaged value was evaluated.

### Measurement of Glass Transition Temperature (Tg)

The curable composition was poured into a mold with a pressing machine and was molded at 185° C. for 10 minutes. The molded article was removed from the mold and was cured at 185° C. for an additional 5 hours. The cured molded article was cut into a size of 5 mm×54 mm×2.4 mm for use as a test piece.

The temperature at which the maximum change in the modulus of elasticity occurred (the largest change in tan δ occurred) in a rectangular tension mode at a frequency of 1 Hz and with a temperature rise of 3° C./min was evaluated as a glass transition temperature using a viscoelasticity measuring device (“RSA II solid viscoelasticity measuring device”, manufactured by Rheometric Corporation).

### Measurement of Linear Expansion Coefficient

The curable composition was poured into a mold with a pressing machine and was molded at 185° C. for 10 minutes. The molded article was removed from the mold and was cured at 185° C. for an additional 5 hours. The cured molded article was cut into a size of 5 mm×5 mm×2.4 mm for use as a test piece.

The linear expansion coefficient in the temperature range of 40° C. to 60° C. was measured twice using a thermomechanical analyzer (“EXSTAR6000 TMA/SS6100” manufactured by Hitachi High-Tech Science Corporation), with a temperature rise of 3° C./min, with a measurement load of 88.8 mN, and in the measurement temperature range of −60° C. to 270° C., and the measured value for the second time was evaluated.

### Measurement of Dielectric Loss Tangent

The curable composition was poured into a mold with a pressing machine and was molded at 185° C. for 10 minutes. The molded article was removed from the mold and was cured at 185° C. for an additional 5 hours. The cured molded article was cut into a size of 1.6 mm×105 mm×1.6 mm for use as a test piece.

The measured dielectric loss tangent at 1 GHz of the test piece subjected to heating vacuum drying and stored in a room at 23° C. and at a humidity of 50% for 24 hours was evaluated in accordance with JIS-C-6481 using an “HP4291B” impedance material analyzer manufactured by Agilent Technologies Inc. in accordance with the following grading system.
A: 0.010 or less
B: More than 0.010 and 0.015 or less
C: More than 0.015 and 0.020 or less
D: More than 0.020

TABLE 1

| | | | | | | | Comparative | |
|---|---|---|---|---|---|---|---|---|
| | | Examples | | | | | Examples | |
| | | 1 | 2 | 3 | 4 | 5 | 1 | 2 |
| Active ester | [parts by mass] | 36.1 | 37.9 | 25.2 | 12.5 | | 50.2 | |
| compound (A-1) | | | | | | | | |
| Active ester (A-2) | [parts by mass] | | | | | 38.3 | | |
| Benzoxazine | [parts by mass] | 18.1 | | | | | | |
| compound (B-1) | | | | | | | | |
| Benzoxazine | [parts by mass] | | 13.2 | 26.2 | 39.1 | 13.1 | | 51.9 |
| compound (B-2) | | | | | | | | |
| Epoxy resin | [parts by mass] | 45.8 | 48.9 | 48.6 | 48.4 | 48.6 | 49.2 | 48.1 |
| Triphenylphosphine | [parts by mass] | 1.0 | 1.0 | 1.0 | 1.0 | 1.0 | 1.0 | 1.0 |
| ICI viscosity (150° C.) | [dPa · s] | 0.8 | 1.1 | 1.3 | 1.4 | 1.9 | 0.9 | 1.6 |
| Gelling time | [s] | 138 | 100 | 107 | 148 | 95 | >600 | >600 |
| Glass transition | [° C.] | 195 | 183 | 211 | 182 | 190 | 136 | 99 |
| temperature (Tg) | | | | | | | | |
| Linear expansion | [ppm] | 50 | 50 | 51 | 53 | 52 | 55 | 60 |
| coefficient | | | | | | | | |
| Dielectric loss tangent | | A | A | B | B | B | A | D |

1. An active ester composition comprising an active ester compound (A) and a benzoxazine compound (B) as essential components.

2. The active ester composition according to claim 1, wherein the active ester compound (A) contains, as an essential component, an active ester compound (A1) which is an esterified product of a compound having one phenolic hydroxyl group in a molecular structure (a1) and an aromatic polycarboxylic acid or an acid halide thereof (a2) or an active ester compound (A2) which is an esterified product of a compound having two or more phenolic hydroxyl groups in a molecular structure (a3) and an aromatic monocarboxylic acid or an acid halide thereof (a4).

3. The active ester composition according to claim 1, wherein the content of the benzoxazine compound (B) is in the range of 0.1 parts to 500 parts by mass with respect to 100 parts by mass of the active ester compound (A).

4. A curable composition comprising the active ester composition according to claim 1 and a curing agent.

5. A cured product of the curable composition according to claim 4.

6. A semiconductor sealing material formed from the curable composition according to claim 4.

7. A printed wiring board formed from the curable composition according to claim 4.
